(19) United States
(12) Patent Application Publication
(10) Pub. No.: US 2010/0324624 A1
(43) Pub. Date: Dec. 23, 2010

(54) AV SYSTEM WITH SKIN CARE AND HEALTH CARE FUNCTIONS

(75) Inventor: Chun-Min Chang, Hsinchu City (TW)

Correspondence Address: Wang Law Firm, Inc., 4989 Peachtree Parkway,, Suite 200, Norcross, GA, 30092, US

(73) Assignee: SynHerb Biotechnology Co., Ltd, Hsinchu City (TW)

(21) Appl. No.: 12/488,529

(22) Filed: Jun. 20, 2009

(51) Int. Cl. A61N 1/00; A61M 21/00

(52) U.S. Cl. 607/46; 607/72; 600/28; 607/50

(57) ABSTRACT

This specification discloses an AV system with the skin care and health care functions. An AV system and a pulse current generator are integrated. A conductor transmits a pulse current output from the pulse current generator to a human body. The pulse current cures or alleviates the user in pain, melancholy, anxiety, and insomnia, accelerates the recovery of wound or bone, and reduces the side effects of radiotherapy and chemotherapy.

### BACKGROUND OF THE INVENTION

1. Field of Invention

The invention pertains to an electronic system related to skin care and health care and, in particular, to an audio/video (AV) system with skin care and health care functions.

2. Related Art

The continuous progress in electronic industry and production techniques, various electronic products have penetrated deep into modern life. This is particularly so for AV devices such as computers, televisions, mobile phones, personal digital assistants (PDA's), radio recorder, DVD player, and walkmans. They have become almost indispensable in daily life. Furthermore, in the trend of miniaturization, many AV products such as mobile phones, PDA's and walkmans are portable.

Although these products have powerful functions, their applications are limited to communications and entertainment. Recently, some people use the communication function of mobile phones for remote medical cares. For example, U.S. Pat. No. 2,008,200,774, U.S. Pat. No. 2,007,255,599, and U.S. Pat. No. 2,007,197,881 suggest using mobile phones to detect health conditions and communicate with health care providers for immediate assistance. Moreover, U.S. Pat. No. 2,005,131,493 uses mobile phones to remote control implanted stimulators.

Nonetheless, all these applications basically use mobile phones to transmit information. They do not directly provide skin care or health care on human bodies. It is thus the purpose of the invention to improve in this direction.

### SUMMARY OF THE INVENTION

An objective of the invention is to provide an AV system with skin care and health care functions that directly provide skin care and health care to human bodies.

To achieve the above-mentioned objective, the invention integrates an AV device with a pulse generator so that they are in electrical connection. The pulse generator has a conductor to be in touch with the human body. A pulse micro current output from the pulse generator goes via the conductor to the human body for skin care and health care purposes.

Since creatures have many ionic compounds, such as amino acids, proteins, fatty acid, nucleic acid, protons, and various kinds of salt, such as potassium, sodium, and calcium that exist in their ionic states. In particular, the potassium, sodium, calcium, and proton are responsible for information transmissions among cells. They have great effects on biological functions. The neural transmissions, muscle contraction, and the regulation of blood filtration and water homeostasis by renal glomeruli all rely on the motion of these ions. A voltage about 20 mV to 200 mV exists across the membrane of a normal cell. Human body cells use the step of depolarization and polarization of the cell membranes to transmit materials and information, thereby achieving various biological functions. The energy factory of an organism, mitochondria, has a voltage of about 200 mV between inside and outside of its inner membrane. The motion of protons can change this voltage to accomplish electron transfers and enhance the activity of ATP synthase. This facilitates the synthesis of ATP.

It is well-known that organisms have biological currents, which affect nutrition, repairs, and regeneration of cells. The biological current in an injured or senescent tissue changes from its normal value. However, if an appropriate micro current is imposed, the biological current of tissue can return to normal. Therefore, the invention uses a pulse current generated by a pulse current generator to induce physiological changes in cells. For example, a pulse micro current of 0.5 mA can accelerate the motion of protons in the mitochondria and increase the activity of ATP synthase. This enhances the ATP production rate by a factor of four as well as the absorption rate of amino acid by cells by 30%˜40%. The protein synthesis rate is also significantly increased. Therefore, the pulse micro current generated by the disclosed pulse current generator can effectively activate cells and increase their energy and tissue renovation ability. Therefore, the microcurrent is effective for skin care. It can be used for beauty and anti-aging. It can reduce wrinkles, improve skin tone and complexion, and remove chloasma and blemishes. Studies also showed significant effects of micro current stimulation on wound healing and bone regeneration. Besides, clinic studies indicate that pulse micro currents can reduce side effects in radiotherapy and chemotherapy. This is particularly effective in alleviating mucositis with symptoms of pain, dysphagia, and dryness.

On the other hand, the pulse micro current can stimulate brain to secrete serotonin and endorphin. As a consequence, the micro current can relieve pressure, elevate the mood, help with sleep, and reduce pains. Some other studies show that the pulse micro current can enhance the alpha waves in the brain, achieving similar effects as Zen meditation and Qi Gong. Therefore, the pulse micro current can be used for depression, melancholia, anxiety, and insomnia treatment. Besides, researches also indicate that the pulse micro current helps with cognitive and muttering disorders, recovering drug addicts and alcoholics, enhancing concentration and memory. Thus, the disclosed AV-system indeed has skin care and health care functions for busy people that face many pressures. The invention can integrate a mobile phone, Walkman, PDA, or some other portable AV device with the pulse current generator into a compact one. It can be further installed on an earphone or glasses. The invention is therefore portable and convenient in use.

On the other hand, the invention can also integrate a computer with the pulse current generator so that users can obtain the above-mentioned benefits of skin care and health care while using the computers.

Other AV electronic appliances, such as radio recorder, DVD player etc. can also be integrated with the pulse generator to obtain the above-mentioned skin care and health benefits.

The method of integrating the AV electronic device and the pulse current generator can build the pulse current generator inside the AV device. They share one circuit board or one power supply. Besides, the pulse current generator can be designed into an IC chip, which can be integrated with the IC chipset of the AV device. The microprocessor module, LCD display, and control module can be integrated to render a beautiful, light, compact, and cheap device.

The method of integrating the AV device and the pulse current generator can have the pulse current generator externally connected to the AV device as well. Common computer ports, such as the universal serial bus (USB), PS/2, Serial (DB-9). ADB (Apple Desktop Bus), AT (Advanced Technology), or some other interface, can be used according to needs. For example, the USB port on a computer can provide a voltage of 5 V and a current of 500 mA. This is sufficient to power the pulse current generator. Other AV devices and output elements can be integrated similarly as well.

Moreover, the disclosed AV device can play medical and health care music with a rhythm in accord with the pulse current generator. The sonic vibrations and melody can further enhance the effects of the pulse current.

On the other hand, the conductor connected to the human body can be designed to have different shapes on the human body end, according to the parts thereof for the convenience of wearing and various special functions. For example, the conductor on the human body end can be a finger cap or glove for the hand, a hat for the head, a mask for the face, a kneecap, wristlet, or elbow pad for the joints, or a waist protection for the waist. Furthermore, the pulse current entering points can be arranged according to the acupuncture point distribution on the human body. For example, Yan Dian on the earlobes, Shi Shuan on the fingers. Yin Tang, ZanZu, Yangbai, Chengqi on the face, Qi Hai and Kuan Yuan on the abdomen, Ming Men on the waist, and Du Bi on the knees are often used acupuncture points. Of course, other acupuncture points on the body can be used according to the disease and purposes.

The frequency of the output pulse current by the disclosed pulse current generator can be high, medium, and low. It is preferably to be low frequencies, smaller than 200 Hz. A frequency corresponding to certain brain wave, eg. beta wave, alpha wave, theta wave or delta wave, can be used to induce brain to a certain state, eg. sleep state, relax state, or allert state etc. The output current of the pulse current generator is preferably smaller than 10 mA.

### BRIEF DESCRIPTION OF THE DRAWINGS

The invention will become more fully understood from the detailed description given herein below illustration only, and thus is not limitative of the present invention, and wherein:

FIG. 1 shows the structure of the invention with a communication AV device;

FIG. 2 is a schematic view of the invention in use, the conductor on the human body end having a clamp shape;

FIG. 3 is a schematic view of the invention in use, the conductor on the human body end having the shape of an earplug of an earphone;

FIG. 4 is a schematic view of the invention in use, the conductor on the human body end having the shape of an earmuff of an earphone;

FIG. 5 is a schematic view of the invention in use, the conductor on the human body end having the shape of a curved hook;

FIG. 6 is a schematic view of the invention in use, the AV device being a computer and the conductor on the human body end having the shape of a glove; and

FIG. 7 is a schematic view of the invention in use, the AV device being a computer and the pulse current generator externally connecting to the USB port of the computer.

FIG. 8-1 is example schematic diagram of pulse generator IC design.

FIG. 8-2 is example schematic diagram of pulse generator IC circuits.

### DETAILED DESCRIPTION OF THE INVENTION

The present invention will be apparent from the following detailed description, which proceeds with reference to the accompanying drawings, wherein the same references relate to the same elements. These descriptions are examples only, and thus is not limitative of the present invention.

The disclosed AV device with skin care and health care functions integrates an AV communication device 10 with a pulse current generator 71.

The AV device 10 can be a mobile phone, walkman, PDA, computer, or some other portable AV device. In this embodiment, the AV device 10 is a mobile phone. It has at least a communication module 11, an input module 21, a liquid crystal display (LCD) and control module 41, a memory 51, and a microprocessor module 61. The communication module 11, a keyboard 21, a power module 31, the LCD and control module 41, the memory 51, and the microprocessor module 61 are electrically connected. The communication module 11 further has an audio frequency encoder 12 so that the communication module 11 is controlled by the microprocessor module 61 to communicate via a speaker/receiver 13.

The pulse current generator 71 can generate a pulse micro current. The pulse current generator 71, the microprocessor module 31 of the AV device 10, and the power module 31 are electrically connected. Of course, the pulse current generator 71 and the AV device 10 can share one circuit board to reduce the production cost and render the device lighter. The pulse current generator 71 can be further extended via a wire with a conductor to be in touch with the human body. The conductor is made of a conductive material, such as conductive rubber, conductive silicon, conductive polymer, conductive carbon powders, conductive silver ink, conductive copper ink, or other conductive metals or alloys. It can be designed to have a shape corresponding to some part of the human body. It transmits the pulse micro current output from the pulse current generator 71 to the human via the wire and the conductor. The pulse micro current output from the pulse current generator 71 can have a high, medium, or low frequency. The frequency of the pulse micro current is preferably smaller than 200 Hz. The output current of the pulse current generator 71 is preferably smaller than 10 mA (milli-Ampere). The best output current is smaller than 1 mA.

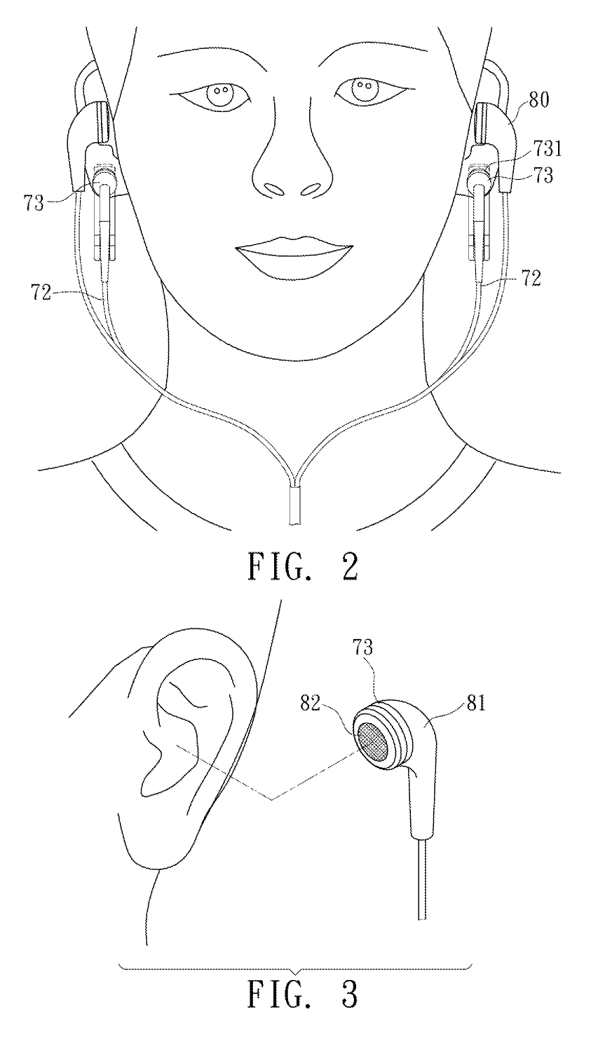

Please refer to FIG. 2 for an embodiment of the disclosed AV device with skin care and health care functions. The AV device has an output unit 80 for making sounds and a wire 72 and a conductor 73 for transmitting the pulse micro current. The conductor on the human body end has a holding part 731 in the shape of a clamp. The conductor 73 can thus be in touch with the skin on the human body via the holding part 731. In this embodiment, the conductor 73 is to hold on to an earlobe for transmitting the pulse micro current to the human body. The sound output unit 80 is an earplug phone.

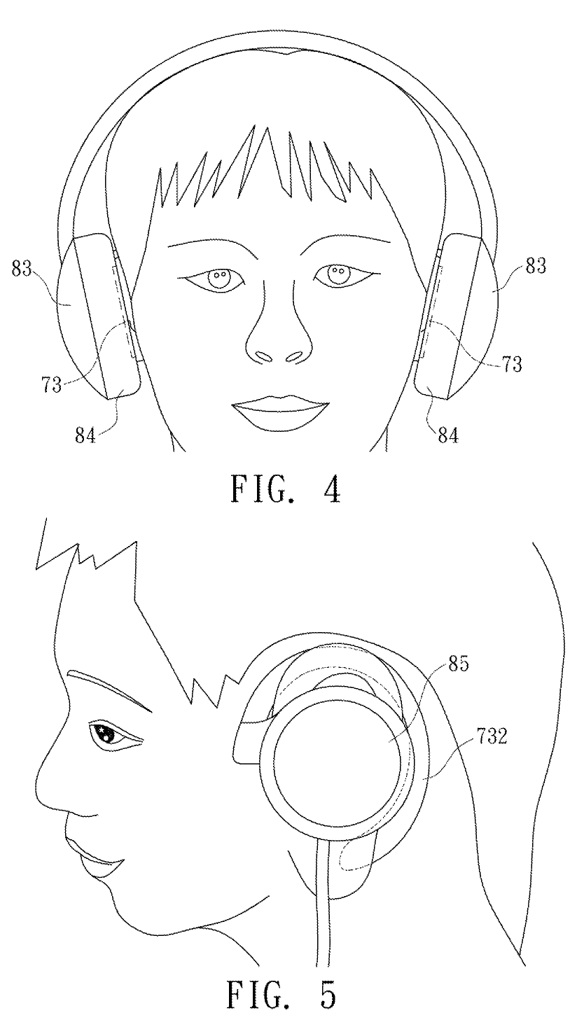

FIG. 3 shows another embodiment of the AV device with skin care and health care functions. The conductor 73 is directly disposed on the surface of the sound output part 82 of an earplug phone 81. When the user put the earplug phone 81 into an ear, the conductor 73 on the surface of the sound output part 82 is in touch with the skin of the canal, thereby transmitting the pulse micro current to the human body. FIG. 4 shows yet another embodiment of the invention. The conductor 73 in this embodiment is installed around the lining of the sound output part 84 of an earmuff phone 83. When the user wears the earmuff phone 83, the conductor 73 around the lining of the sound output part 84 is in touch with the human ear, transmitting the pulse micro current to the human body.

Please refer to FIG. 5 for yet another embodiment of the invention. The conductor 73 is mounted on an earphone 85, bending and extending to form a curved hook part 732. The earphone 85 is then hung on the human ear with the hook part 732 formed by the conductor 73. The hook part 732 formed by the conductor 73 is then in touch with the skin on the back of the ear, thereby transmitting the pulse micro current to the human body.

It should be particularly mentioned that the conductor of the invention can be designed to have different shapes for different parts of the human body. For example, it can be made to have the shape of a finger cap or glove for the hand, a hat for the head, a mask for the face, a kneecap, wristlet, or elbow pad for the joints, or a waist protection for the waist. All such variations should be included in the scope of the invention.

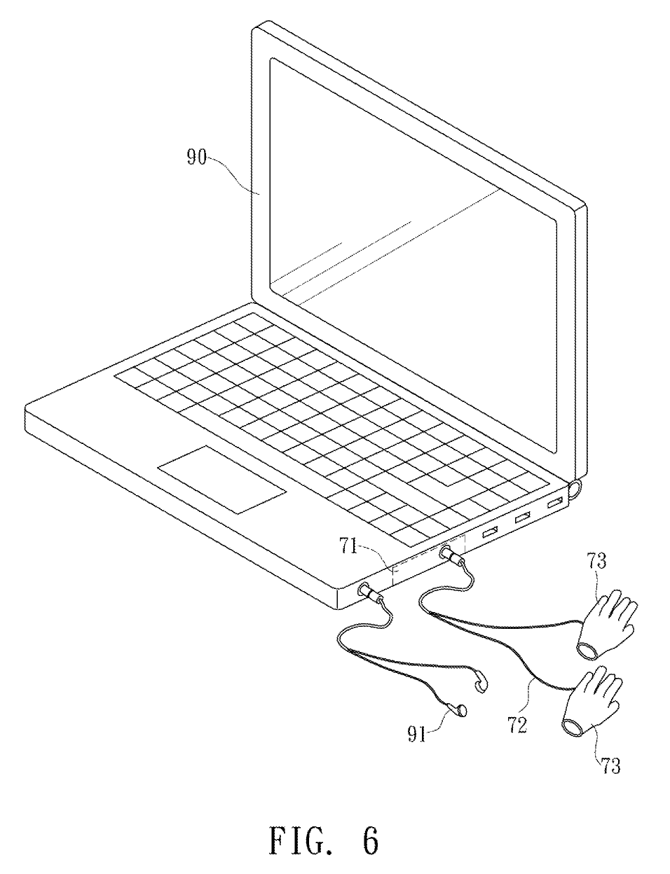

Please refer to FIG. 6 for a further embodiment of the invention. The AV device is a computer 90, with the pulse current generator 71 installed therein. The computer 90 is connected with an earphone 91 for making sounds and a wire 72 and a conductor 73 for transmitting the pulse micro current generated by the pulse current generator 71. The conductor 73 on the human body end has the shape of a glove corresponding to the human hand. When the user wears the glove, the conductor 73 transmits the pulse micro current to the human body. The user can use the earphone 91 to listen to music at the same time. The music, in particular, can have a special melody for skin care and health purposes.

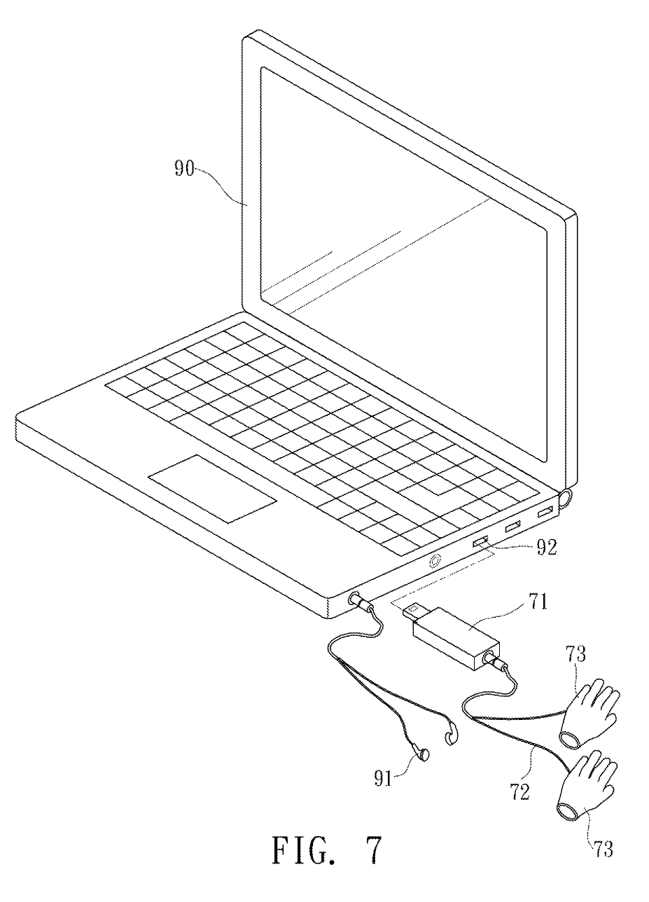

Yet another embodiment of the invention is illustrated in FIG. 7. The AV device is a computer 90. The pulse current generator 71 is externally connected to the USB port 92 of the computer 90. The pulse current generator 71 has a wire 72 and a conductor 73 for transmitting the pulse micro current. When the user wears the glove made of the conductor 73, the pulse micro current is transmitted to the human body via the conductor 73.

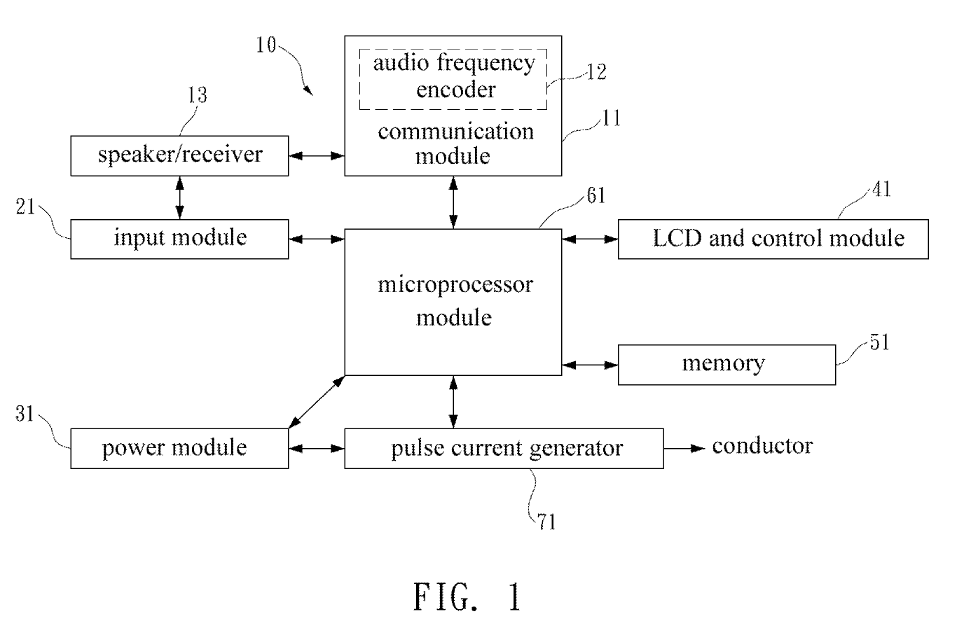

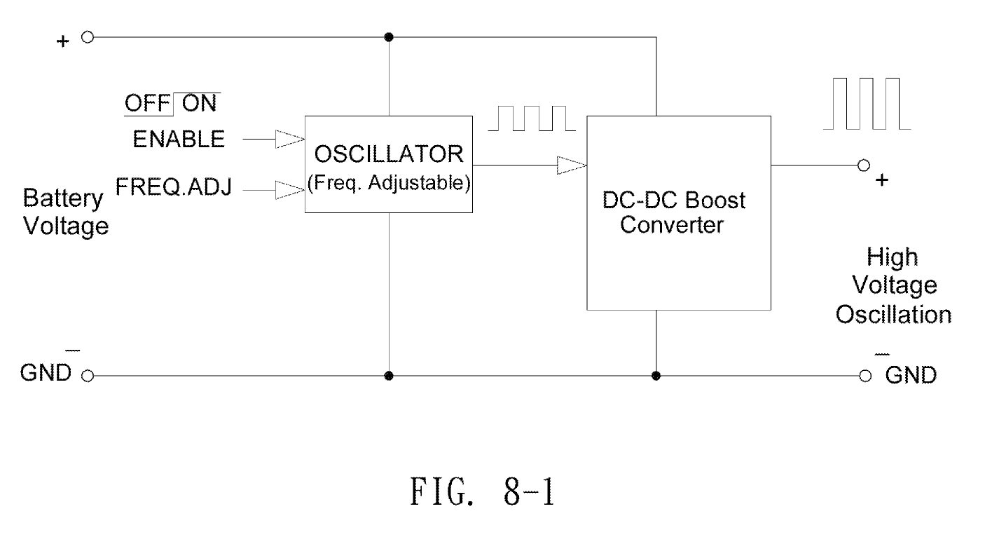

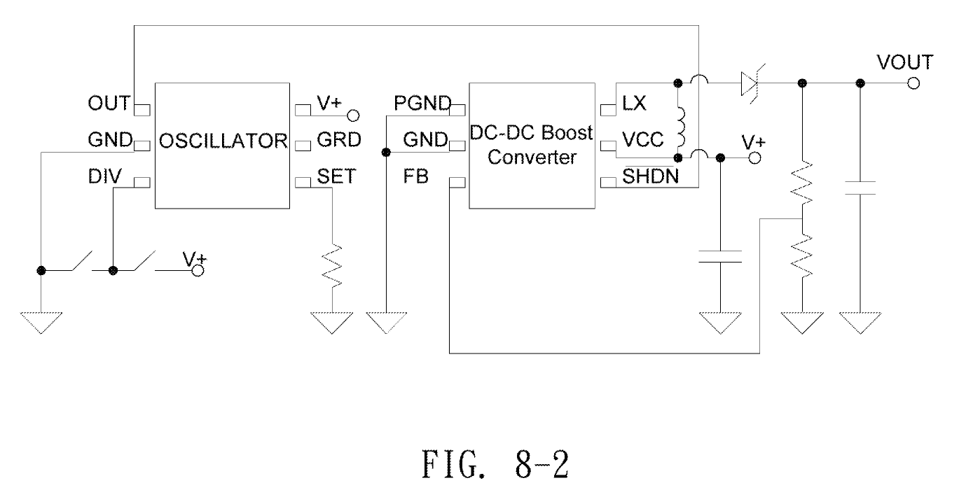

To make the system more compact and more cost effective, the pulse current generator can be an IC chip. FIG. 8-1 and 8-2 show the example schematic diagrams of pulse generator IC design and circuits. The DC-DC boost converter is optional, depending on the voltage of the battery and the output current requirement. This pulse current generator chip can be further integrated with the chipset of the AV device.

Human Body Test 1: Test of Anxiety Relaxation.

Testees are interviewed and given the Spielberger State-Trait Anxiety Inventory (STAI) test. Those who get scores higher than 50 points are considered have anxiety. There are totally 12 people randomly divided into a treatment group and a control group, each of which has 6 people. This is a single-blind test. The testees first listen to soft music for 20 minutes. Afterwards, the testees' earlobes are hung with the disclosed ear-holding device. The current for the treatment group is set at 1 Hz and 0.6 mA. This electrical current is so small that the testees almost feel nothing. After 20 minutes, the testees remove the ear holder and are then given the STAI again. The new scores and the old scores are compared. The control group also goes through the same procedure, except that the pulse current generator does not generate any electrical current. The result is that the average score of the treatment group drops from 52.4 to 32.1. The testees obviously feel better and happier, with less anxiety than before the test. The average score of the control group drops slightly from 53.1 to 50.2, statistically no effect at all.

Human Test 2: Test of Pain Relief

The test gather 18 people with ages between 22 and 75 that feel chronic pain, muscle stiffness, and fatigue. Among them, 10 people have been diagnosed to have fibromyalgia. The McGill pain score of these 18 people has an average of 30±4. These people are randomly divided into a treatment group and a control group, each of which has 9 people. This is a double-blind test. Testees are asked to put on the ear-holding device every day for one hour. The treatment group receives a feelingless pulse current of 0.5 Hz and 0.2 mA, while the control group receives none. Various tests are given after three weeks. The result is that the average McGill pain score of the treatment group drops to 21±3. They also have a better mood and feel that the pain has been relieved. The average McGill pain score of the control group stays at 29±5. The endorphin concentration in the blood of the treatment group has an obvious increase (P<0.01) to as high as 98%.

Human Body Test 3: Test of Alleviating Insomnia

Fifteen patients who think they have insomnia use the disclosed system at home. After 2 weeks, eleven people indicate that they can fall asleep more easily. Nine of them feel that the sleep quality has improved. Four of them think that it is useless.

Human Body Test 4: Test of Alleviating Depression

Testees are given the Geriatric Depression Scale (GDS) test. People with scores above 5 are considered to be depression patients. In this test, there are 16 patients with an average score of 7.3. The testees are asked to put on the disclosed ear-holding device on their earlobes. The strength of the pulse current is adjusted to a comfortable one, between 0.2 mA and 2 mA. The subjects are treated two times a day, each taking 40 minutes. They are analyzed by the GDS test three weeks later. The average score of the treatment group drops from 7.3 to 3.8. The testees apparently feel happier and more positive. A similar improvement is also observed in the Zung Self-rating Depression Scale test. Moreover, the serotonin concentration in their blood is obviously increased (P<0.01).

Human Body Test 5: Test of Accelerating Wound Recovery

The test gathers 10 diabetes patients whose feet have ulcer wounds difficult to recover. After using the disclosed device with the pulse current of 2 Hz and 1 mA around the wounds, two times a day for two weeks and 30 minutes each time, the wounds have obvious improvements.

Human Body Test 6: Test of Reducing Side Effects of Radiotherapy

After Co-60 radiotherapy, 8 cancer patients experienced the side effect of xerostomia that causes dry mouth, ulcer, inflammation, and difficulty in swallowing food. The discomfort of dry month can be divided into five levels. The first level is normal. The fifth level is the most serious, completely unable to swallow food. Using the invention on the auricle for 20 minutes, the discomfort drops from level 3˜4 to level 1˜2 on the average. The discomfort of dry mouth was considerably improved when the user uses it every day.

Human Body Test 7: Test of Reducing Wrinkles

12 women of age between 35 to 75 were enrolled for skin care tests. A facial mask with micro-current conducting points disposed on accupoints was used for this test. After moisturized with herbal essence, the facial mask was put on face. A pulse current stimulation of 1 Hz, 0.5˜1 mA was applied for 20 minutes daily. After 2 weeks, all testees reported a reduction of wrinkles, improved skin tone and complexion by self-evaluation.

In summary, the invention integrates an AV device with a pulse current generator. A conductor sends the pulse micro current generated by the pulse current generator to the human body. As a result, the user can get benefits on beauty and health. The user can be cured or alleviated in pains, depression, anxiety, insomnia, etc due to the pulse current. It also accelerates the recovery of wounds and injured bones, as well as reduces side effects of radiotherapy and chemotherapy. The disclosed AV device with skin care and health care functions can be miniaturized for carrying, so that the user can use it at any time anywhere.

Although the invention has been described with reference to specific embodiments, this description is not meant to be construed in a limiting sense. Various modifications of the disclosed embodiments, as well as alternative embodiments, will be apparent to people skilled in the art. Therefore, it is contemplated that the appended claims will cover all modifications that fall within the true scope of the invention.

What is claimed is:

1. An audio/video (AV) system with skin care and health care functions, comprising an AV device, a pulse current generator, and a conductor wherein the AV device electrically connects to a pulse current generator and has a conductor to be in touch with a human body, and the pulse current output from the pulse current generator goes via the conductor to the human body.

2. The AV system with skin care and health care functions of claim 1, wherein the pulse current generator provides a pulse current at a frequency less than 200 Hz.

3. The AV system with skin care and health care functions of claim 1, wherein the pulse generator provides a pulse current smaller than 10 mA.

4. The AV system with skin care and health care functions of claim 1, wherein the AV device is a communication device.

5. The AV system with skin care and health care functions of claim 4, wherein the AV communication device is a mobile phone, walkman, or a personal digital assistant (PDA).

6. The AV system with skin care and health care functions of claim 1, wherein the AV device is a computer.

7. The AV system with skin care and health care functions of claim 1, wherein the AV device is an earphone or glasses.

8. The AV system with skin care and health care functions of claim 1, wherein the AV device is a television, radio recorder or DVD player.

9. The AV system with skin care and health care functions of claim 1, wherein the pulse generator is built inside the AV device or connected to the AV device externally.

10. The AV system with skin care and health care functions of claim 9, wherein the external pulse generator connects to the AV device using a universal serial bus (USB), PS/2, serial DB-9, Apple desktop bus (ADB), or advanced technology (AT) interface.

11. The AV system with skin care and health care functions of claim 1, wherein the conductor on the human body end is designed to be worn at an appropriate portion of the human body.

12. The AV system with skin care and health care functions of claim 11, wherein the conductor on the human body end has a structure of earplug, earmuff, hook or clamp.

13. The AV system with skin care and health care functions of claim 11, wherein the conductor on the human body end has a structure of finger cap, glove, hat, mask, wristlet, kneecap, elbow pad for the joints, or waist protection.

14. The AV system with skin care and health care functions of claim 1, wherein the AV device includes an element to play health care music.

15. The AV system with skin care and health care functions of claim 1 that cures or improves depression, melancholia, insomnia, or anxiety.

16. The AV system with skin care and health care functions of claim 1 that accelerates the recovery of wounds or injured bones.

17. The AV system with skin care and health care functions of claim 1 that cures or alleviates pain, fibromyalgia syndrome, side effects of chemotherapy or side effects of radiotherapy.

18. The AV system with skin care and health care functions of claim 1 that cures or improves drug addicts, alcoholics, or enhances concentration and memory.

19. An IC chip which can generate a pulse current and is integrated with the chipset of an AV device, wherein the AV system has skin care and health care functions.

20. A method of using an AV system with skin care and health care functions comprising the step of delivering a pulse current generated by the AV system into a human body through an acupuncture point.
